(19) United States
(12) Patent Application Publication
(10) Pub. No.: US 2022/0330711 A1
(43) Pub. Date: Oct. 20, 2022

(54) REDUCED ENVIRONMENTAL IMPACT TEXTILE PRODUCTS HAVING PEELABLE LAYERS

(71) Applicant: Peel Away Labs, Inc., Jersey City, NJ (US)

(72) Inventors: Maxwell COHEN, New York, NY (US); Howard BRAUNER, Manalapan, NJ (US)

(21) Appl. No.: 17/640,524

(22) PCT Filed: Sep. 3, 2020
(86) PCT No.: PCT/US2020/049165

§ 371 Date: Mar. 4, 2022

Related U.S. Application Data
(60) Provisional application No. 62/896,813, filed on Sep. 6, 2019.

(51) Int. Cl. A47C 31/10; A47G 9/02; A41D 31/02; A41D 31/102; A47C 27/00; A47C 31/11

(52) U.S. Cl. CPC A47C 31/105; A47G 9/0253; A41D 31/02; A41D 31/102; A47C 27/006; A47C 31/113; A41D 2400/72; A41D 2400/52; A47G 2400/10; A47G 2400/02

(57) ABSTRACT

Provided are protector articles, comprising: a plurality of sheets overlaid one to the next, the plurality of sheets comprising a bottommost sheet, a sheet comprising a comfort face and a barrier face, the comfort face comprising a biodegradable spunlace nonwoven material, the barrier face comprising a water-resistant biodegradable material, and at least one of the plurality of sheets being releasable (e.g., releasably attached) to the article. The articles can be used in bedding, apparel, and furniture.

### RELATED APPLICATIONS

The present application claims priority to and the benefit of U.S. patent application No. 62/896,813, “Reduced Environmental Impact Textile Products Having Peelable Layers” (filed Sep. 6, 2019), the entirety of which application is incorporated herein by reference for any and all purposes.

### TECHNICAL FIELD

The present disclosure relates to the field of layered textiles.

### BACKGROUND

There are an estimated over 7.4 million college freshmen and sophomore students in the United States alone. Every year, new students enter the system, and there is a constant influx of students.

College students are, however, known for a carefree lifestyle and many do not adhere to the cleanliness standards they had at home. This is due to a parent, house cleaner or other person taking care of the students' needs before college.

One of the needs is the changing of sheets that requires the student to take everything off the bed, take off the fitted sheet and take it to the laundry to wash. It can take hours to wash and dry, bring the sheets back to the dorm and remake the bed. Or, the student can send their laundry out which can take a week to get back, so the student needs a second set of bed sheets. It is no surprise that some students ultimately do not wash their sheets all semester.

A dorm (college, high school or ever summer camp) is a hotbed for germs and dirt. Most students not only sleep in their bed, but use it for a workspace, kitchen table, drinking pub, guest lounge and guestroom for romantic encounters and a napping place. Food, dirt and bodily fluids cover the sheets, but they continue to use them without cleaning.

There are other situations and other people where changing sheets can be problematic. For example, babies often soil their sheets and those sheets need to be changed frequently. Changing such a sheet might be problematic if, for example, there isn't a second caregiver at home to hold the baby while the first caregiver changes the sheet.

Similarly, it is common for elderly and/or incontinent people to soil their sheets. In many cases, the elderly person is infirmed or otherwise unable to leave the bed easily. Additionally, people recovering from surgical procedures or hospital stays can have wounds that can dirty sheets requiring the sheets to be changed.

As a result, it would be beneficial to have a bedsheet which can more easily and rapidly be replaced than presently occurs. It would be further beneficial for these sheets to be concurrently waterproof or water resistant so as to protect the underlying mattress and any other materials laid on the mattress. By having a self-waterproof sheet, the speed to replace a sheet improves dramatically.

Similarly, the materials used for an easily replaceable and waterproof bed sheet could be used for pillowcases or blanket covers as well.

It would be beneficial to overcome the problem of excessive time and effort necessary to replace a bedsheet or comparable item, excessive time and effort to clean up possible damage, and to overcome the problem of damaged mattresses thereby shortening the mattress life and incurring unnecessary costs.

### SUMMARY

The present disclosure relates generally to a layered article, such as but not limited to a sheet set, a pillowcase, a blanket cover, a mattress protector, a bib, or an apron; and methods of manufacturing a layered mattress protector. The present disclosure also relates generally to a layered pillow protector and methods of manufacturing a pillow protector.

The present disclosure solves the aforementioned problem by providing a multi-layered peel-away article (which can be termed a “sheet set”) that is comprised of a plurality of layered sheets, which can be attached with perforations (or otherwise attached) so as to facilitate easy removal, to reveal a new, fresh, clean layer without having to go through the efforts of cleaning sheets or attaching a new sheet to the bed. That is, a single sheet system may have a plurality of sheets (or pillowcases or blanket covers) arranged together in a way such that when one sheet needs to be replaced (such as due to soiling), that sheet can be removed without a new sheet having to be applied. Further, the sheet can be removed without, for example, moving an elderly patient or a baby entirely off of the surface of the bed. In one embodiment, a top or uppermost sheet can be peeled away from the remainder of the sheet set. (Intermediate sheets can also be removed, if desired.)

The sheets can be sized for use on a porta-crib, crib, twin, double, queen, king, California king, hospital bed size or even dog bed sized/shaped. Similarly, the sheet set can be other bedding, such as mattress covers, pillowcases, blanket covers and the like. The fabric may be dyed any color and can be printed with any logo, such as a sports team, college name or even dates for reminders when to change the sheets (e.g. October 1, October 15, November 1) or to purchase new sheets (e.g., when there are only two or sheets remaining), as well as with patterns and designs.

An advantage of the peelable, multi-layered, waterproof function of the product is when the top layer becomes wet or soiled, is it can be easily removed, and disposed of, so that a fresh layer can be exposed and then used by the consumer.

In one embodiment, the present disclosure is directed to a sheet set, where each sheet in the sheet set has a “comfort” side comprising a non-irritating surface, and an opposing waterproof side for serving the additional purpose of a mattress protector. In the event the sheet is soiled (such as due to incontinence) the upper (or exposed) sheet of the plurality of sheets can be removed readily and the mattress remains protected and need not be cleaned off. For an apron or bib, the waterproof side can be either the comfort side or the exposed side.

According to an aspect of the present disclosure, a layered mattress protector and sheet is provided, comprising a bottom layer including a fitted portion; a plurality of layers attached to said bottom layer, each of said attached layers having a top side and a bottom side and a waterproof material of the bottom side of each; wherein the plurality of layers can be collectively stitched along both of the long edges to the bottom layer; and wherein each of the layers in the plurality of layers has at least one perforation located proximal to the stitching. In other embodiments, the stitching may not be included. In yet other embodiments, there may be no perforations other than those caused by the stitching. In still other embodiments, each of said plurality of layers may have perforations and include extensions such that each could wrap around the mattress corners and themselves serve as fitted sheets.

According to another aspect of the present disclosure, a method of manufacturing a layered mattress protector and sheet is provided.

In one embodiment, each of the plurality of sheets could including an unperforated tab to facilitate removal of each sheet; and wherein the plurality of sheet is in a stacked arrangement such that each of the sheet has a greater (or lesser) area than a sheet located immediately below or be equal in size.

In one embodiment, the present disclosure provides protector articles, comprising: a plurality of sheets overlaid one to the next, the plurality of sheets comprising a bottommost sheet, a sheet comprising a comfort face and a barrier face, the comfort face comprising a biodegradable spunlace nonwoven material, the barrier face comprising a water-resistant biodegradable material, and at least one of the plurality of sheets being releasable (e.g., releasably attached) to the article.

Also provided are methods of using the disclosed articles, including disposing the articles over a mattress, a pillow, a seat, and the like.

Further provided are methods, comprising: forming an article that comprises a plurality of sheets overlaid one to the next, a sheet comprising a comfort face and a barrier face, the comfort face comprising a biodegradable non-woven spunlace material, the barrier face comprising either a water-resistant or water repellent or water proof biodegradable material, at least one of the plurality of sheets optionally being releasably attachable to the article.

Also provided are protector articles, comprising: a plurality of sheets overlaid one to the next, a sheet comprising a comfort face and a barrier face, the comfort face comprising a nonwoven biodegradable spunlace material derived from cellulose and a polysaccharide, the barrier face comprising a water-resistant biodegradable material, the water-resistant biodegradable material optionally being porous, and at least one of the plurality of sheets being releasably attached to the article; and a sleeve, a strap, a band, a skirt having an adjustable perimeter and optionally comprising a biodegradable elastic material, or any combination thereof.

These and other features, aspects and advantages of the present disclosure will become better understood with reference to the following description and claims.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various aspects discussed in the present document, and any dimensions given in the drawings are illustrative only and are not-limiting. In the drawings:

FIG. 1 depicts a layered mattress protector according to the present disclosure;

FIG. 2 depicts a layered mattress protector according to the present disclosure;

FIG. 3 depicts a layered mattress protector according to the present disclosure;

FIG. 4 depicts a layered mattress protector according to the present disclosure;

FIG. 5 depicts a layered mattress protector according to the present disclosure;

FIG. 6 depicts a layered mattress protector according to the present disclosure;

FIG. 7 depicts a layered mattress protector according to the present disclosure;

FIG. 8 depicts a layered mattress protector according to the present disclosure;

FIG. 9 depicts a layered mattress protector according to the present disclosure;

FIG. 10 depicts a layered mattress protector according to the present disclosure.

FIG. 11 depicts a layered mattress protector according to the present disclosure.

FIG. 12 depicts a layered mattress protector according to the present disclosure.

FIG. 13 provides a layered mattress protector according to the present disclosure; and

FIG. 14 provides a layered mattress protector according to the present disclosure.

### DETAILED DESCRIPTION OF ILLUSTRATIVE EMBODIMENTS

The present disclosure is directed to a multi-layered sheet set product with a plurality of removable, peelable layers sized for use as bedding with beds sized such as but not limited to porta crib, crib, changing table, toddler bed, twin-size bed, twin XL-size bed, full-size bed, queen-size bed, king-size bed, or California King-sized bed. Alternatively, the multi-layer product of the present disclosure is usable as bedding, an apron, a bib, etc. In another embodiment, the present disclosure is directed to a multi-layered mattress protector with removable, peelable or removable layers in the same size. In another embodiment, the present disclosure is directed to a multi-layered car seat cover with removable layers. In another embodiment, the present disclosure is directed to a multi-layered baby bib with removable, peelable layers. In another embodiment, the present disclosure is directed to a multi-layered cooking apron with removable, peelable layers.

In general, the present disclosure is directed to a multi-layer product, where each layer has a top side comprising a material, materials, and/or a surface pleasing to the intended use—e.g., cotton or flannel for use as a bed sheet—and an opposing side comprising a material, materials, and/or a surface conformant to other possible uses—e.g., waterproof materials for protecting a mattress or item of clothing. In addition, each layer in the multi-layer product is similarly constructed and the exposed or outer layer can easily be removed, for example, once its useful life has completed.

It should be understood that the disclosed articles can transport body heat away from the user and can also absorb perspiration from the person, e.g., via wicking. The article can be configured to draw perspiration or other moisture away from the user.

In general, the multi-layer product of the present disclosure is comprised of a plurality of layers, including a base layer and a plurality of upper layers, in which each of the plurality of upper layers may be removable one at a time.

In general, the base layer is a part of a larger assembly. For example, the base layer of an apron may include elements for attaching to a person. In another example, the base layer of a sheet or mattress cover may include the fitting sections for surrounding and attaching to a mattress as a fitted sheet would. In another example involving a sheet, the base layer may include or be attached to bed decorative borders.

In general, the product of the present disclosure includes a plurality of upper layers which are collectively attached to the base layer in some way such that each of said upper layers can be individually removed for discarding. Each layer can be attached using breakaway stitching or include perforations for tearing, or some combination. Typically, the aforementioned stitching or perforations might be included on less than all edges.

In any of the aforementioned embodiments, the product can be formed of non-woven (spunlace) fabric. A spunlace fabric can have a parallel or cross grain pattern, though this is not a requirement. A spunlace fabric can be, e.g., a nonwoven product made via entangling a web of loose fibers through jets of water (or via other means), which process entangles the fibers. In some embodiments, the spunlace fabric has isotropic properties, e.g., has the strength in any direction.

A spunlace fabric can have a density of, e.g., about 20 grams per square meter or greater.

In another embodiment, the product is formed of woven fabric. In another embodiment, the product can be formed of a combination of non-woven and woven fabrics. The material used can be comprised of one of or a blend of cotton, bamboo, rayon, lyocell, hemp, flax or any other fiber that can be made into a non-woven or woven material.

A sheet of the present disclosure can include a waterproof (or water-resistant) membrane, preferably laminated, with a minimum thickness of 0.001 mm that is either breathable (micro-porous) or non-breathable, on at least a portion of one side. The laminated membrane is on the bottom side of the fabric. In one embodiment, the membrane is affixed to the fabric by either heat or a gluing process, although the membrane can also be affixed to the fabric via stitching, static attraction, or other methods. In some embodiments, the membrane can be affixed to the fabric via friction or a Velcro™-type (hook-and-loop or hook-and-hook) interaction. In some embodiments, the membrane and fabric can be separable from one another by a user, so as to facilitate different forms of disposal for each. The membrane can be made from at least one of polyethylene, polyurethane, poly vinyl chloride, polybutylene, latex, silicone or any other waterproofed material, either alone by itself or in combination with any other named or inferred material that is assembled into a multi-layered finished product; the membrane can also be made from a biodegradable material. (Suitable such biodegradable materials are described elsewhere herein.) In some embodiments, the fabric and the membrane are made from different biodegradable materials; in some embodiments, the fabric and the membrane are made from the same base biodegradable material but with different properties (e.g., porosity, thickness, permeability).

In a sheet set, the waterproof layer can be the layer facing the mattress and can be referred to herein as the “under” side. The laminated member of the present disclosure can extend to the entirety of a sheet or can just extend to a portion of the sheet, depending upon need, cost, and manufacturability. For example, a sheet in the plurality of sheets might have edge stitching for connecting to a base sheet, and the laminate member can extend only to the stitching.

The other side, or “over” side, is formed of materials appropriate to use, such as cotton, flannel, paper, or other material, for a bedsheet.

Particularly when used as bedding, the article of the present disclosure includes sheets with two sides. The side closest to the mattress is intended to provide for a water barrier so as to protect the mattress from spills, soiling, and the like. That same side also has the added feature of comparatively high friction in that it is intended not to slip or shift relative to the mattress. The other side, or the side away from the mattress and the side which will come in contact with a person, is intended to be formed of a material to provide comfort to the person. Non-limiting examples are cotton and cotton flannel. The two sides of each sheet are bonded together to form a single sheet, where the bonding is accomplished through heat or gluing. However, the bonding is intended not to adversely impact the benefits described above.

In one embodiment, the product is multi-layer, and each layer is individually peelable. The layers are combined together either by stitching with needle and thread, or such as by using a hook and loop system, such as Velcro™, a zipper, and/or a removable adhesive, either alone or in combination. The layers can be married to a bed skirt, or to a solid fabric, that can be made of any material, that can have elastic attached to allow for a form fit or an edge made of any material to complete the sheet, apron, baby bib or any other desired item, such as but not limited to one directed to protecting a surface.

With regard to the peel away attributes of the present disclosure, the goal is for a user, with limited effort, to separate the exposed sheet from the remaining sheets. To accomplish this goal, each success sheet is attached to the remainder of the sheet set in such a way so that the sheet remains functional (if, for example, used as a sheet, it should be comfortable to sleep on), yet is easily removable.

The attachment can be by means of adherence (such as by gluing or heating) or stitching, as examples. Known alternatives can alternatively be used. In an alternate embodiment, the attachment can be by stitching. Stitching can be accomplished on a sheet by sheet basis or collectively with one stitching.

In at least some embodiments, the stitching can be used in combination with perforations. For a user to remove each sheet the sheet will tear off at the perforation. In at least some embodiments, one or more tabs can be included on each sheet to facilitate removal.

In an alternative embodiment, the stitching and the material together are of the form of “breakaway” stitching, wherein a layer can be removed by a user when pulling at the location of the stitching, and the layer is torn away from the remainder of the article.

In an embodiment with perforations, the perforations will extend on two opposing sides only. That is, for a bedsheet, the perforations would typically be on the two long sides of the bed. The perforations could be located along a line denoting the edge between the top and the side of the mattress, it could be along the line denoting the edge between the bottom and the side of the mattress, or it could be elsewhere. Wherever it might be, a remnant might be left behind. For an apron, the perforation might only be on one side. Once torn away, removed layer of the present disclosure is discardable.

In at least some embodiments, the removable layers are sized identically to one another and to the base layer. In other embodiments, the size of the removable layers can vary (becoming either smaller or larger as they become more proximal to the base layer). In some embodiments, the removable layers are generally sized for only the top of the mattress, whereas the base layer is sized comparably to a fitted sheet.

The figures included herein are directed to only some of the embodiments described herein.

The following detailed description includes various modes of carrying out the disclosure. The description is not to be taken in a limiting sense but is made merely for the purpose of illustrating the general principles of the disclosure. When dimensions are included, the reader should view these as exemplary only and not limiting in any way.

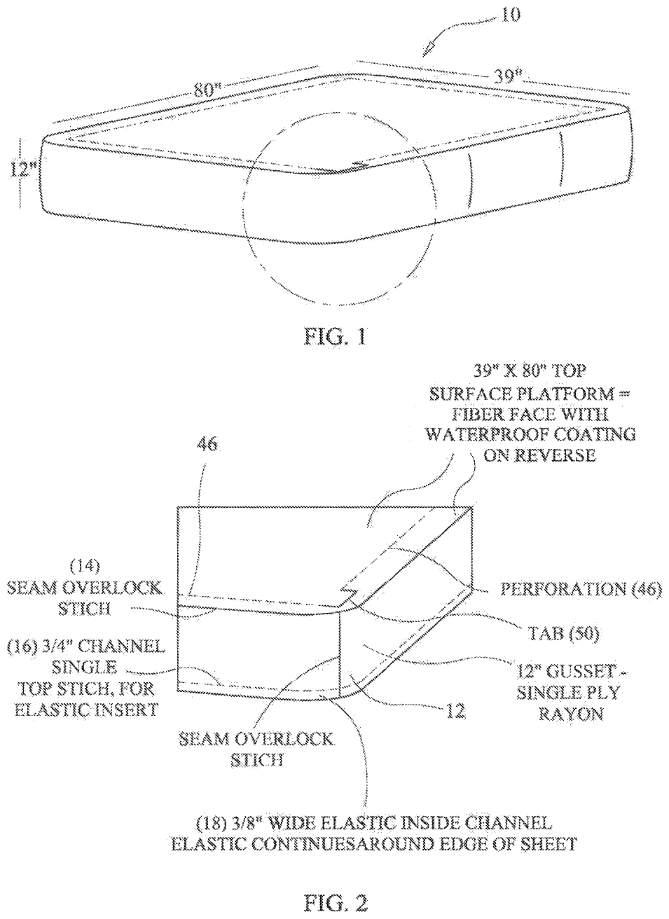

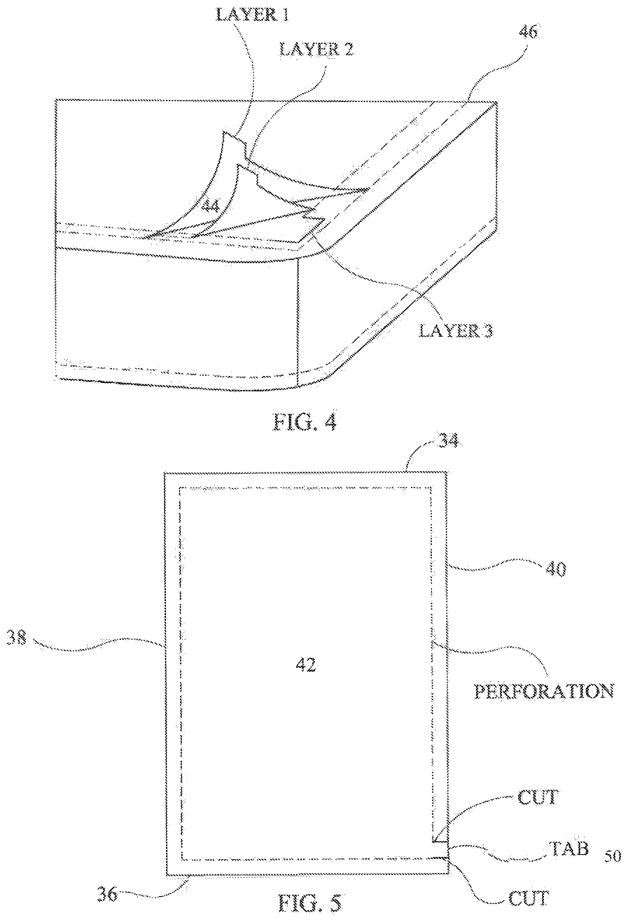

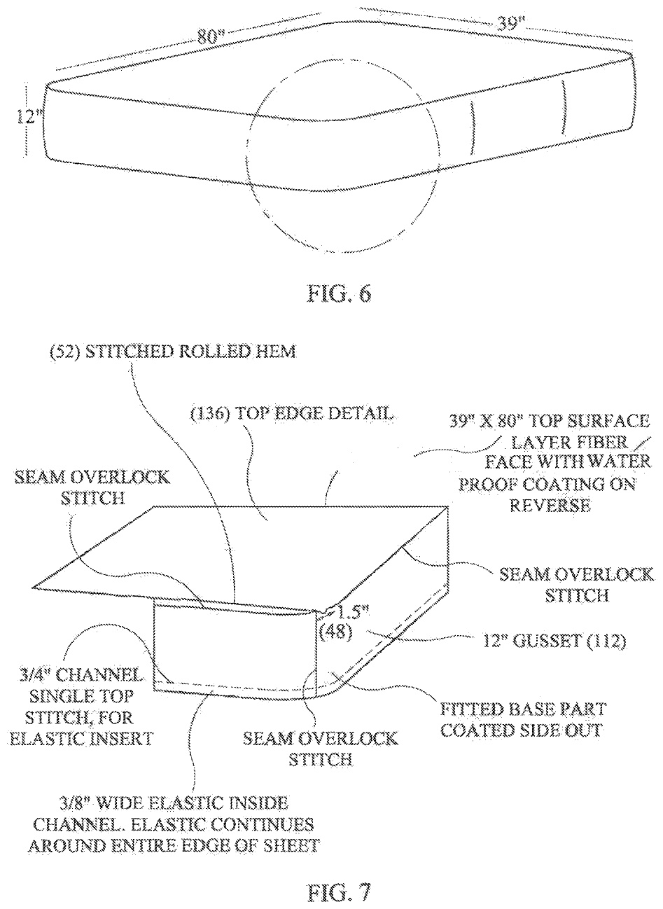

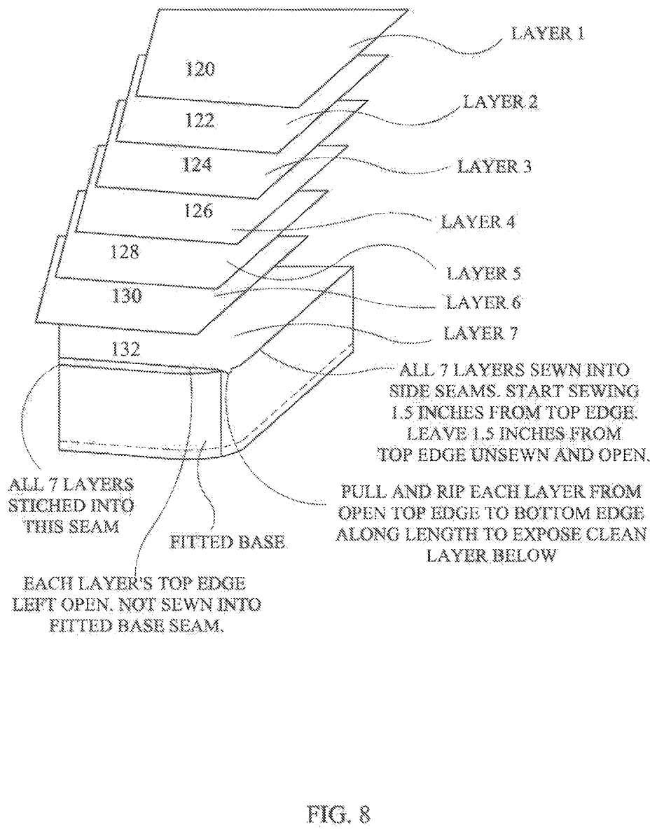

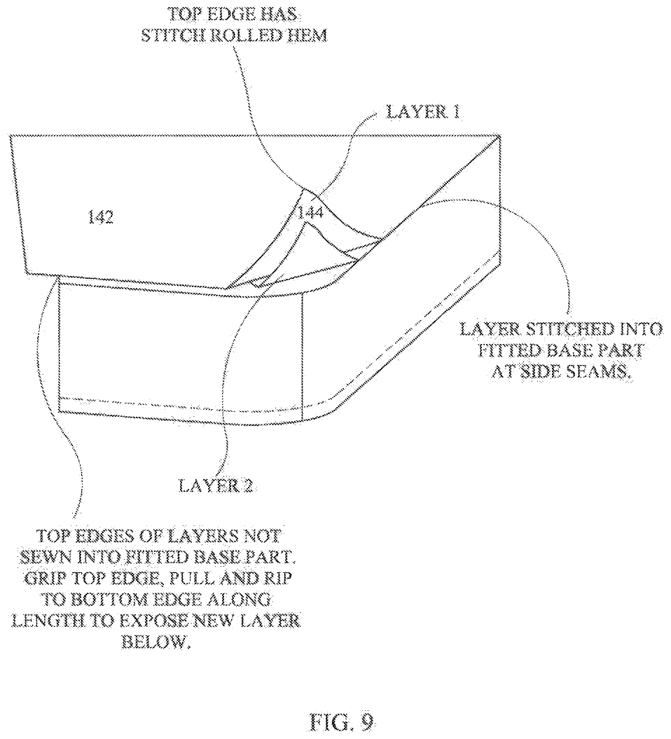

With reference to exemplary, non-limiting FIGS. 1-12, the present disclosure provides a layered mattress protector and sheet (10). It is noted that two embodiments are shown. FIGS. 1-5 depict one embodiment having a perforation that runs substantially parallel to the top side (34), bottom side (36), right side (40) and left side (38) and includes a tab (50). FIGS. 6-8 depict an embodiment in which there are only two perforations, that are perforation seams (80), that are substantially parallel to the right side and left side in each of at least three fitted sheet layers. They are also slightly inside the right side and left side of each of the at least three fitted sheet layers, which is to say they are located closer to the center point of each of the at least three fitted sheet layers.

The layered mattress protector and sheet can have a bottom fitted portion (12, 112); at least three fitted sheet layers (e.g. 20, 22, 24, 26, 28, 30, 32, 120, 122, 124, 126, 128, 130 and 132). While there can be any number of fitted sheet layers, according to one embodiment there are seven layers. Each fitted sheet layer has a top side (34, 134), bottom side (36, 136), right side (40, 140) and left side (38, 138) and an upper portion (42, 142) and lower portion (44, 144). Note that the numbers for the different sides are shown for one layer of the many layers of the fitted sheet layers. Each layer can have the same sides. There can be a waterproof material on the lower portion (44, 144) of each of the at least three fitted sheet layers. (It should be understood that a waterproof material can be a coating or a layer, as these terms are treated as interchangeable.)

The upper portion of each fitted sheet layer can be, e.g., a biodegradable spunlace material. Then at least three fitted sheet layers are stitched along the right side (40, 140) and left side (38, 138) to the bottom fitted portion (12, 112) and the top side (34, 134) and bottom side (36, 136) are unstitched. Each of the at least three fitted sheet layers has at least one perforation (46, 146).

In one exemplary embodiment, depicted in FIGS. 1-5, there is a single perforation that runs substantially parallel to the top side (34), bottom side (36), right side (40) and left side (38) and includes a tab cutout portion (50). The term perforation can be a series of small holes or other gaps that allows for the fitted sheet layers to be removably attached, it can also be a perforation seam, that is a seam that is stitched creating holes, or perforations, yet having the added stability of a seam. The perforation (46, 146 also referred to as a perforation seam) creates a portion of the fitted sheet layer that is smaller than the entire fitted sheet layer that can be easily removed to reveal another, new fitted sheet layer below it. There is also a waterproof material (e.g., a membrane) on the lower portion (44, 144) of each of the at least three fitted sheet layers. The at least three fitted sheet layers are stitched along the right side (40, 140) and left side (38, 138) to the bottom fitted portion (12, 112) and the top side (34, 134) and bottom side (36, 136) are unstitched. Each of the at least three fitted sheet layers has at least one perforation (46, 146) that is at least substantially parallel to the right side and left side in each of at least three fitted sheet layers.

The at least three fitted sheet layers can be stitched along the right side (40, 140) and left side (38, 138) to the bottom fitted portion (12, 112) along a portion of the right side (40, 140) and a portion of the left side (38, 138) to leave an unstitched portion (48) along a portion of each end of the right side and the left side. The unstitched portion (48) along a portion of each end of the right side and the left side can be between one and three inches, e.g., one and a half inches. This allows for the easy removal of each layers of the at least three fitted sheet layers. It is not required that the unstitched portion (48) be along both sides, it can be along either a portion of the right side (40, 140) or a portion of the left side (38, 138). It should be towards one end of the right (right side (40, 140) or the left side (38, 138). The term end is intended to denote the area close to the top side (34, 134) or the bottom side (36, 136). The unstitched portion (48) along a portion of at least one end of the right side and the left side can be between one and three inches, e.g., one and a half inches.

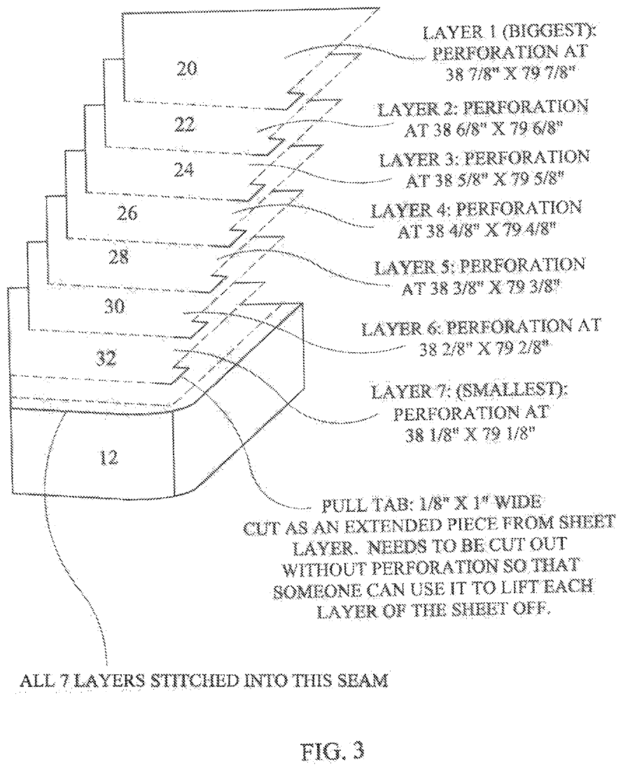

The upper portion (42) of each fitted sheet layer can be a biodegradable spunlace material, e.g., rayon. The fitted sheet layers can be all the same size. According to an alternate embodiment, each of the at least three fitted sheet layers is slightly larger than the layer that is below it. As shown in FIG. 3, fitter sheet layer (20) is the biggest and has a perforation at 38⅞×29⅞, fitted sheet layer (22) is slightly smaller and has a perforation at 38 6/8×29 6/8, fitted sheet layer (24) is slightly smaller and has a perforation at 38⅝×29⅝, fitted sheet layer (26) is slightly smaller and has a perforation at 38 4/8×29 4/8, fitted sheet layer (28) is slightly smaller and has a perforation at 38⅜×29⅜, fitted sheet layer (30) is slightly smaller and has a perforation at 38 2/8×29 2/8 and fitted sheet layer (32) is slightly smaller and has a perforation at 38⅛×29 ⅛. As shown, the perforated portion of a sheet can be larger than the layer that is below it.

It should be understood that a given sheet may be attached (e.g., via adhesion, via stitching) to the sheet beneath it. Alternatively, a sheet may be attached (e.g., via adhesion, via stitching) to the base layer (described elsewhere herein).

A perforated portion can have a tab cutout portion (50). The tab cutout portion (50) can be between a portion of the perforated portion (46) and at least one of the top sides (34), bottom side (36), right side (40) and left side (38) of the at least three fitted sheet layers. According to one embodiment, the tab cutout portion (50) is away from the top side (34). This is to prevent inadvertent removal of a layer and any possible discomfort to the user. The top side (34) can be sewn with a rounded stitch that edges the top side to provide an edged top side. The top side (34) can also be stitched to provide a stitched rolled hem top side (54). There can also be a printed item (56) on the upper portion of the at least three fitted sheet layers. The printed item (56) can be a school logo, or it can be, by way of example, say “October” as a reminder to pull off the layer at the end of October. Of course, it could say October 1, the next layer October 15, the next layer November 1, etc. In this way, the user is reminded of when to change their sheets. An article can also include other text on a given sheet, e.g., text to remind the user to purchase their next sheet set, and the like. Different sheets can comprise different adornments, e.g., different sheets can be of different colors and can feature the logos of different sports teams, different story characters, and the like.

Another aspect of the present disclosure provides a method of method of manufacturing a layered mattress protector and sheet, comprising: providing a bottom fitted portion (12, 112); providing at least three fitted sheet layers (e.g. 20, 22, 24, 26, 28, 30, 32, 120, 122, 124, 126, 128, 130 and 132), each fitted sheet layer having a top side (34, 134), bottom side (36, 136), right side (40, 140) and left side (38, 138) and an upper portion (42, 142) and lower portion (44, 144), wherein the lower portion (44, 144) of each of the at least three fitted sheet layers has a waterproof material; providing at least one perforation (46, 146) that is at least substantially parallel to the right side and left side in each of at least three fitted sheet layers; stitching the at least three fitted sheet layers along the right side and left side to the bottom fitted portion and leaving the top side and bottom side unstitched. The step of stitching the at least three fitted sheet layers along the right side (40, 140) and left side (38, 138) to the bottom fitted portion (12, 112) and leaving the top side (34, 134) and bottom side (36, 136) unstitched can be further comprising the step of: stitching along the right side and left side to the bottom fitted portion to leave an unstitched portion (48) along at least one end of the right side and the left side. The unstitched portion (48) along at least one end of the right side and the left side is between one and three inches, e.g., one and a half inches. Each of the at least one perforations (46, 146) of the at least three fitted sheet layers can be slightly larger than the layer that is below it. There can be the step of providing a tab cutout portion (50). The tab cutout portion (50) can be along at least one of the top side, bottom side, right side and left side of the at least three fitted sheet layers. There can be the step of sewing the top side with a rounded stitch that edges the top side to provide an edged top side. There can be the step of stitching the top side to provide a stitched rolled hem top side. There can also be the step of printing a printed item (56) on the upper portion of each of the at least three fitted sheet layers.

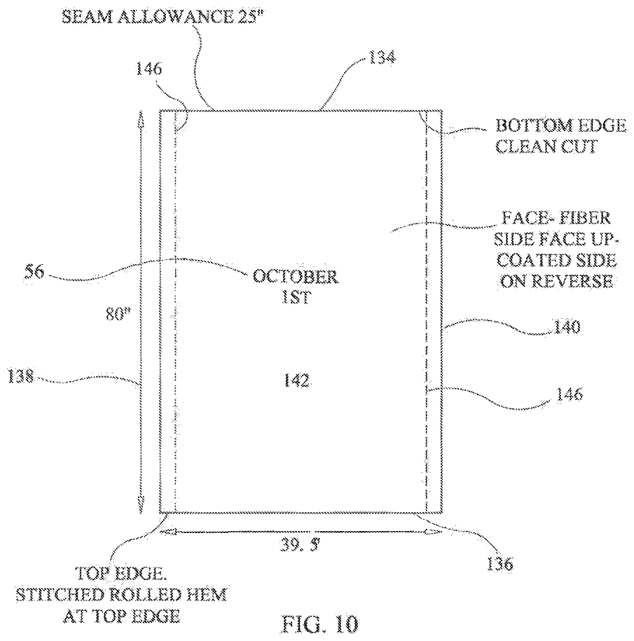

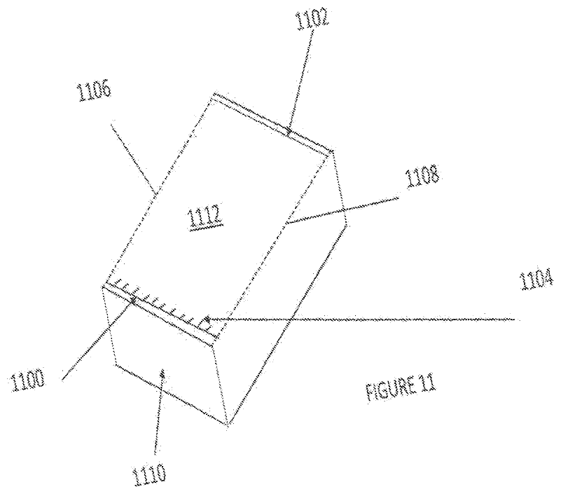

FIGS. 10, 11 and 12 depict a layered mattress protector according to the present disclosure.

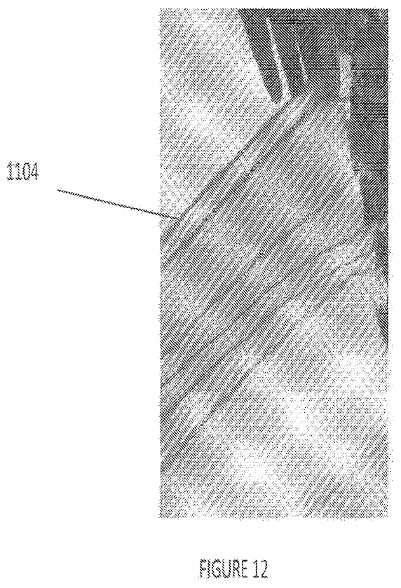

FIG. 11 depicts an embodiment of the present disclosure. A layered mattress protector and sheet is provided with a bottom fitted portion (1110); at least three fitted sheet layers (1112), each fitted sheet layer having a top side (1100), bottom side (1102), right side (1108) and left side (1106) and an upper portion (1112) and lower portion (not seen in FIG. 11, would be the portion opposing the upper portion (1112) on the underneath of the three fitted sheet layers). Also note that the at least three fitted sheet layers are layered, so FIG. 11 only depicts one of the layers, but there are at least three fitted sheet layers, as depicted in other figures. There is a waterproof material on the lower portion of each of the at least three fitted sheet layers (1112). The at least three fitted sheet layers (1112) are stitched along the right side (1106) and left side (1108) to the bottom fitted portion (1110) the stitch creating at least one perforation seam and the top side (1100) and bottom side (1102) are unstitched to the bottom fitted portion (1110) leaving open ends; and the top side (1100) is an edged top side (1104). FIG. 12 depicts a picture of the edged top side of each of the at least three fitted sheet layers (1112) according to the present disclosure and the top side being unstitched.

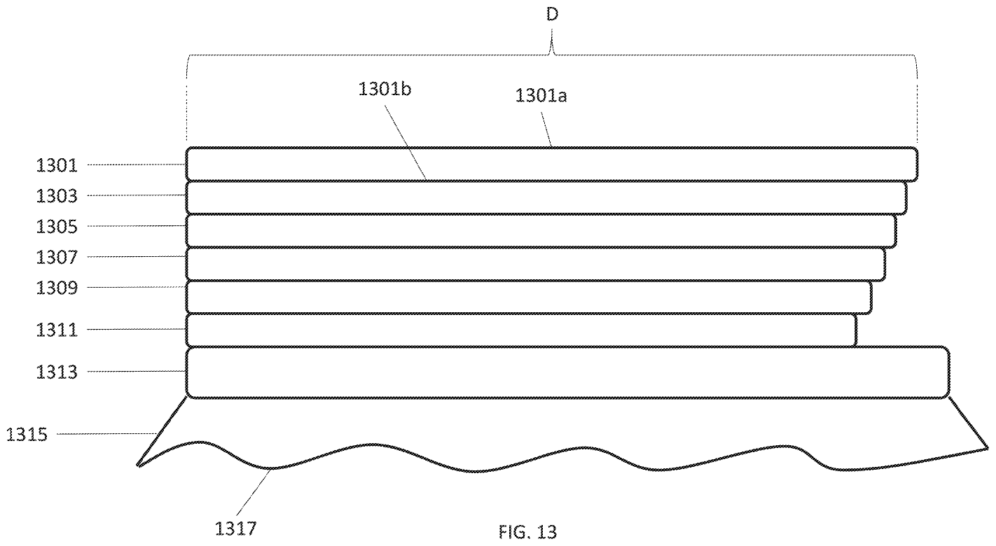

FIG. 13 provides a cutaway view of an article according to the present disclosure. As shown, an article can include base layer 1313 and sheets 1301, 1303, 1305, 1307, 1309, and 1311, which sheets are disposed atop one another. As shown, sheet 1301 includes comfort face 1301a and barrier face 1301b. (Sheets 1303, 1305, 1307, 1309, and 1311 also comprise their own comfort faces and barrier faces, which are not labeled here). A sheet can be removably attached to an adjacent sheet. For example, sheet 1301 can be attached (e.g., via gluing, Velcro™) to sheet 1303. When the user desires a fresh sheet, the user simply lifts/tears sheet 1301 away from sheet 1303 so as to expose sheet 1303. A sheet can include a tab, a loop, a pull-ring, or other feature to enhance a user's ability to securely grip the sheet in order to remove the sheet. It is not a requirement that a given sheet be attached to another sheet, as sheets can be laid atop one another and maintain their relative positions (e.g., without sliding) by virtue of the friction between adjacent sheets.

As shown in FIG. 13, a sheet can define a cross-sectional dimension (e.g., width or length) D. The dimension D can vary from sheet to sheet; as shown, uppermost sheet 1301 has a larger dimension D than does sheet 1303 that is directly beneath sheet 1301. This is not a requirement. It should also be understood that although not shown in FIG. 13, a given sheet can extend beyond base layer 1313.

As shown in FIG. 13, an article can also include a skirt 1315, which skirt includes an attachment portion 1317. Attachment portion 1317 can be, e.g., an elasticized region, a corded/tied portion, and the like, and can be used to secure the article to a mattress.

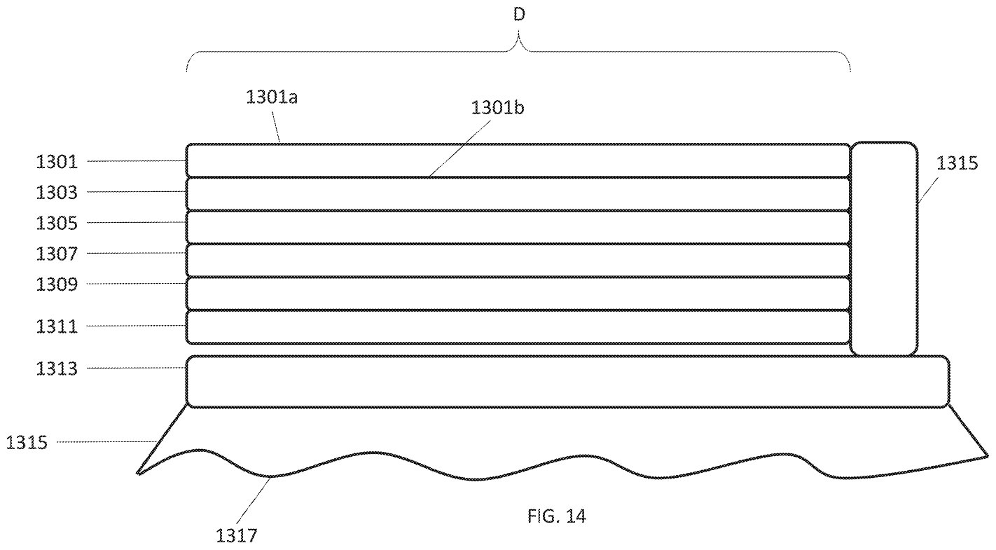

FIG. 14 provides a cutaway view of an article according to the present disclosure. As shown, an article can include base layer 1313 and sheets 1301, 1303, 1305, 1307, 1309, and 1311, which sheets are disposed atop one another. As shown, sheet 1301 includes comfort face 1301a and barrier face 1301b (Sheets 1303, 1305, 1307, 1309, and 1311 also comprise their own comfort faces and barrier faces, which are not labeled here).

A sheet can be attached (e.g., via a perforated region) to trunk 1315; trunk 1315 can be attached to or even be a part of base layer 1313. One or more of sheets 1301, 1303, 1305, 1307, 1309, and 1311 can be releasably attached to trunk 1315 such that when a sheet is removed, the sheet is torn away from trunk 1315. A sheet can include a tab, a loop, a pull-ring, or other feature to enhance a user's ability to securely grip the sheet in order to remove the sheet. It is not a requirement that a given sheet be attached to another sheet, as sheets can be laid atop one another and maintain their relative positions (e.g., without sliding) by virtue of the friction between adjacent sheets. Sheet 1311 can be attached (independently) to base layer 1313.

As shown in FIG. 14, an article can also include a skirt 1315, which skirt includes an attachment portion 1317. Attachment portion 1317 can be, e.g., an elasticized region, a corded/tied portion, and the like.

### Embodiments

It should be understood that the following relates to illustrative, non-limiting embodiments of the disclosure and that modifications can be made without departing from the spirit and scope of the disclosure as set forth in the following claims.

Embodiment 1. A protector article, comprising: a plurality of sheets overlaid one to the next, the plurality of sheets comprising a bottommost sheet, a sheet comprising a comfort face and a barrier face, the comfort face comprising a biodegradable spunlace nonwoven material, the barrier face comprising a water-resistant biodegradable material, and at least one of the plurality of sheets being releasable (e.g., releasably attached) to the article.

By “biodegradable” is meant a material whose chemical and physical characteristics undergo deterioration and completely degrade when exposed to microorganisms, aerobic, and anaerobic processes. Examples are polymers that degrade through the action of living organisms, light, air, water and combinations of the foregoing. Such polymers include a range of synthetic (biodegradable) polymers, e.g., polysaccharides/cellulose, such as paper.

Biodegradation reactions are typically enzyme-catalyzed and generally occur in the presence of moisture. Natural macromolecules containing hydrolyzable linkages, such as protein, cellulose and starch, are generally susceptible to biodegradation by the hydrolytic enzymes of microorganisms. A few man-made polymers, however, are also biodegradable. The hydrophilic/hydrophobic character of polymers greatly affects their biodegradability, with more polar polymers being more readily biodegradable as a general rule. Other important polymer characteristics that affect biodegradability include crystallinity, chain flexibility and chain length.

As described elsewhere herein, the plurality of sheets can comprise sheets that are all of the same size. The plurality of sheets can also comprise sheets of different sizes. As an example, the plurality of sheets can comprise sheets that are ordered from largest to smallest. The plurality of sheets can also comprise sheets that are ordered from smallest to largest. A given sheet can vary in length and/or thickness from a sheet that is above or below the given sheet. For example, a top sheet in a stack may be longer and/or wider than the sheet beneath the top sheet. A given sheet can also vary in composition from a sheet that is above or below the given sheet. The bottommost sheet can be of the same composition and/or size of any sheet in the plurality of sheets, although this is not a requirement.

Embodiment 2. The protector article of Embodiment 1, further comprising a sleeve (e.g., a sewn sleeve), a strap, a band (e.g., an elastic or non-elastic band), a skirt (optionally having an adjustable and/or elastic perimeter), or any combination thereof. Any of the foregoing (e.g., a skirt) can optionally comprise a biodegradable elastic material. Similarly, any of the foregoing can also comprise a biodegradable non-elastic material. Any of the foregoing can be used to secure the protector article to an object or user being protected, e.g., to a bed, to a chair, or to other furniture.

As one example, a protector article can include an elasticized skirt that is sized to secure the article to a mattress, such as a United States extra-long twin mattress, a United States queen size mattress, a United States king size mattress, a United States double size mattress, and the like. Alternatively, a protector article can include a biodegradable material (e.g., a cord or a lace) that can be tied, tightened, or otherwise used (e.g., using a fastener, such as a cord-lock, clip, or other similar fastener) to secure the article to an object or user being protected. An article according to the present disclosure can include a Velcro™-type strap, loop, or other such fastener that can be used to secure the article to an object or user.

Embodiment 3. The protector article of Embodiment 2, wherein the sleeve, the strap, the band, the skirt, or any combination thereof is secured to the bottommost sheet.

Embodiment 4. The protector article of Embodiment 1, further comprising a base layer. A base layer can be, e.g., a water-resistant (e.g., waterproof) membrane or layer, such as a vinyl, polyurethane, or other such material. A base layer can include cushioning, e.g., quilting.

Embodiment 5. The protector article of Embodiment 4, further comprising a sewn sleeve, a strap, a band, a skirt (optionally having an adjustable perimeter), or any combination thereof.

Embodiment 6. The protector article of Embodiment 5, wherein the sleeve, the strap, the band, the skirt having an adjustable perimeter, or any combination thereof is secured to the base layer.

Embodiment 7. The protector article of any one of Embodiments 1-6, wherein at least two of the plurality of sheets are secured to one another by sewing, gluing, hook-and-loop fastening, snaps, clamping, buttons, heat-sealing, ultra-sonic welding, or any combination thereof.

Embodiment 8. The protector article of any one of Embodiments 1-7, wherein at least one of the plurality of sheets is attached to the protector article via one or more perforated linkages or one or more stitched linkages.

Such a linkage can be tearable by the user. As described elsewhere herein, a perforated linkage can be one whereby the perforations are tearable by the user such that the user can, by tearing the linkage, remove one (e.g., the topmost) of the plurality of sheets to as to expose the sheet beneath the torn-away sheet.

A linkage can run along some or all of the perimeter of a sheet. As an example, a given rectangular sheet can be secured to the article via a perforated region that runs the length of one of the long sides of the sheet such that the sheet in question is secured to the article along only one edge. This is of course only one possible embodiment, as a sheet can be secured to the article via, e.g., by a single L-shaped perforation line, by two separate perpendicular perforation lines, by one or more straight perforation lines, and by one or more curved perforation lines. Sheets can be secured to the article via perforation lines that are not in register with one another. As an example, an article can be constructed such that sheets are attached to the article via tearable lines that even sheets are attached along their right-hand edges and odd sheets are attached along their left-hand edges, in an alternating fashion. It should be understood that an article can be constructed such that one or more sheets are tearably attached to a common “trunk” portion of the article, e.g., in the manner of the tearable pages of a laboratory notebook. Sheets can be secured by alternating linkages, e.g., whereby a first sheet is secured by a linkage at one end (e.g., the head) of the sheet, and a second sheet is secured by a linkage at an opposite end or edge (e.g., the foot or bottom) of the sheet.

As another example, a first sheet can be secured by a linkage at the upper edge (e.g., the head) of the sheet. A second sheet can be secured by a linkage along the right hand edge of the sheet. A third sheet can be secured by a linkage along the bottom of the sheet. A fourth sheet can be secured by a linkage along the left edge of the sheet. A fifth sheet can be secured by a linkage along the top edge of the sheet, and so on. Successive sheets can be secured by different edges, e.g., top, then right, then bottom, then left, then top; or top, then bottom, then right, then left, then top; or top, then left, then bottom, then right, then top, then left; or top, then left, then right, then bottom, then top, and so on. Successive sheets can thus be secured by linkages along the same edge for each sheet, or different edges for successive sheets. In this way, a stack of sheets can remain relatively level as successive sheets are removed.

A protector article according to the present disclosure can be constructed such that the protector article can stretch lengthwise and/or widthwise. The stretchability can be, e.g., up to about 15% lengthwise (e.g., up to 1%, 2%, 3%, 4%, 5%, 6%, 7%, 8%, 9%, 10%, 11%, 12%, 13%, 14%, 15%, and all intermediate values and ranges) and/or up to about 15% (e.g., up to 1%, 2%, 3%, 4%, 5%, 6%, 7%, 8%, 9%, 10%, 11%, 12%, 13%, 14%, 15%, and all intermediate values and ranges)widthwise. Without being bound to any particular theory, stretchability can allow for a comparatively even pressure distribution of the body on the sheet by “giving” in the Horizontal direction. This allows for maximum comfort. In some embodiments, a protector article has a greater stretch in the width (minor) direction than the length (major) direction. For example, an article may have a stretch of up to about 10 or even 15% in the width direction, and a stretch of 5% or less in the length direction.

Embodiment 9. The protector article of any one of Embodiments 1-7, wherein at least one of the plurality of sheets is attached to the protector article via one or more adhesive linkages, one or more stitches, hook-and-loop fasteners, snaps, buttons, or any combination thereof.

Embodiment 10. The protector article of any one of Embodiments 1-9, wherein at least a portion of the biodegradable spunlace nonwoven material is characterized as plant-derived. By “plant-derived” is meant formed directly from plant material as well as formed from materials that originate in plants.

Embodiment 11. The protector article of Embodiment 10, wherein at least a portion of the biodegradable spunlace nonwoven material is derived from one or more of corn, seaweed, agave, hemp, or any combination thereof. The spunlace nonwoven material can be derived from virtually any plant.

A variety of materials can be used in the spunlace nonwoven material. Some non-limiting examples include polylactic acid, e.g., BIO-FLEX™, available from FKuR Kunststoff GmbH, Germany; ECOLOJU™, available from Mitsubishi Plastics, Inc., Japan; HYCAIL™, available from Hycail, the Netherlands; INGEO 2002D™, available from NatureWorks LLC, Minnetonka, Minn.); polyhydroxybutyrate (e.g., BIOMER L™, available from Biomer, Germany); polyvinyl alcohol (e.g., BIOSOL™, available from Panteco, Italy; GOHSENOL™, available from Nippon Gohsei, Japan; MAVINSOL™, available from Panteco, Italy; MOWIOL™, available from Kuraray America, Inc., Houston, Tex.; KURARAY POVAL™, available from Kuraray America, Inc., Houston, Tex.); polybutylene succinate (e.g., GREEN PLASTICS™, available from Mitsubishi, Japan); polyhydroxyalkanoates (e.g., MIREL™, available from Telles (Metabolix and Archer Daniels Midland Company), Lowell, Mass.); polycaprolactones (e.g., CAPA™, available from Solvay, United Kingdom); ECOFLEX™, available from BASF, Germany); starches (e.g., BIOPLAST™, available from Biotec, Germany; BIOPAR™, available from BIOP Biopolymer Technologies AG, Dresden, Germany; CEREPLAST COMPOSTABLES™ and CEREPLAST HYBRID RESINS™, available from Cereplast, Hawthorne, Calif.; COHPOL™, available from VTT Chemical Technology, Finland; ECOPLAST™, available from Groen Granulaat, the Netherlands; EVERCORN™, available from Japan Corn Starch Co., Japan; MATER-BI™, available from Novamont, Italy; PLANTIC™, available from Plantic Technologies Limited, Victoria, Australia; SOLANYL™, available from Rodenburg Polymers, the Netherlands; SORONA™, available from DuPont, Wilmington, Del.; RE-NEW ™, available from StarchTech, Golden Valley, Minn.; TERRATEK™, available from MGP Ingredients, Atchison, Kans.; VEGEMAT™, available from Vegeplast, France); celluloses (e.g., BIOGRADE™, available from FKuR Kunststoff GmbH, Germany); and other biopolymers (e.g., LUNARE SE™, available from Nippon Shokubai, Japan), and combinations thereof. The foregoing are exemplary only; MATER-BI™ is considered particularly suitable.

Embodiment 12. The protector article of Embodiment 10, wherein at least a portion of the biodegradable spunlace nonwoven material comprises polycaprolactone, polysaccharide (e.g., starch), polylactic acid, or any combination thereof.

Embodiment 13. The protector article of any one of Embodiments 1-9, wherein the biodegradable spunlace nonwoven material comprises one or more of cotton, viscose, bamboo, hemp, flax, or any combination thereof. Essentially any at least partially natural fiber can be used in the biodegradable spunlace material. Synthetic fibers (that are biodegradable) can also be used.

Embodiment 14. The protector article of any one of Embodiments 1-13, wherein the comfort face is characterized as having a GSM of from about 18 to about 60 g/m². The comfort face can have a GSM of from about 18 to about 60 g/m², from about 20 to about 58 g/m², from about 22 to about 56 g/m², from about 24 to about 54 g/m², from about 26 to about 52 g/m², from about 28 to about 50 g/m², from about 30 to about 48 g/m², from about 32 to about 46 g/m², from about 34 to about 44 g/m², from about 36 to about 42 g/m², from about 38 to about 40 g/m², or even from about 34 to about 48 g/m².

Embodiment 15. The protector article of any one of Embodiments 1-14, wherein the comfort face is characterized as comprising at least a first fiber population and a second fiber population, the first fiber population differing from the second fiber population in at least one of size and composition. The first fiber can be any naturally derived material including cotton, rayon tencel, bamboo, hemp or any other natural fiber with the second fiber being bamboo, to accomplish a blend of 80-90% rayon or other natural fiber with 10-20% bamboo.

Embodiment 16. The protector article of any one of Embodiments 1-15, wherein the comfort face is characterized as having a static coefficient of friction of from about 0.01 to about 1.5. The static coefficient can be from, e.g., about 0.01 to about 1.5, from about 0.03 to about 1.4, from about 0.05 to about 1.2, from about 0.10 to about 1.0, from about 0.25 to about 0.75, from about 0.30 to about 0.6, from about 0.4 to about 0.5, or even from about 0.4 to about 0.45, or even from about 0.01 to about 0.4. The static coefficient of friction can be from about 0.2 to about 0.5, or even from about 0.3 to about 0.4. In one exemplary embodiment, the comfort face has a static coefficient of friction of 0.38.

The comfort face can have a kinetic coefficient of friction of from about 0.01 to about 0.50, from about 0.05 to about 0.4, from about 0.1 to about 0.35, from about 0.2 to about 0.3, or even from 0.21 to 0.30. In one exemplary embodiment, the comfort face has a static coefficient of friction of 0.26.

Embodiment 17. The protector article of any one of Embodiments 1-15, wherein the comfort face is characterized as having a softness. For example, the comfort face can have a Furikami value of, e.g., from about 3 to about 6, or from about 3 to about 5, or even from about 3.5 to about 4.5. The comfort face can have a THV value of, e.g., from about 3.1 to about 3.6. The comfort face can have a Numeri value of, e.g., from about 3.3 to about 5.7.

Embodiment 18. The protector article of any one of Embodiments 1-17, wherein the barrier face is characterized as either solid or porous. By porous is meant pervious to vapor, whether accomplished via pores (straight or non-straight) formed through a solid material or via passages formed in a fabric, e.g., via the interstitial spaces between fibers in a woven or nonwoven material.

Embodiment 19. The protector article of any one of Embodiments 1-18, wherein the barrier face is characterized as having a waterproof rating of from about 0 to about 50,000, a breathability of from about 0 to about 30,000 g/m2/day, or any combination thereof.

Barrier faces can also be characterized in terms of their waterproof rating. Waterproof rating can be defined as the height (in mm) of a column of water above a 1″×1″ square of material at which the water begins to leak through the material (per the JIS-L 1092 method). The higher the number, the more waterproof the material under examination. In some embodiments, the barrier face has a waterproof rating of from 0 to about 5,000 mm. The barrier face can additionally have a waterproof rating of from about 10,000 to about 15,000 mm. The barrier face can have a waterproof rating of from about 15,000 to about 20,000. In some embodiments, the barrier face can have a waterproof rating of more than about 20,000, e.g., from 20,000 to 50,000, from 20,000 to 40,000, or even from 20,000 to 30,000 mm. The barrier face can have a waterproof rating of from 0 to about 50,000.

In one example test, various volumes of water were placed (at ambient conditions) on the uppermost sheet of a plurality of sheets according to the present disclosure, and the water penetration (if any) to the second and third layers was tested. The results of this testing are provided below:

TABLE 1
Exemplary water resistance test results

| Volume of | | | |
|---|---|---|---|
| water for test | Testing period | Second layer | Third layer |
| 250 ml | 24 hours | No water | No water |
| | | penetration | penetration |
| | | observed | observed |
| 250 ml | 48 hours | No water | No water |
| | | penetration | penetration |
| | | observed | observed |
| 500 ml | 24 hours | No water | No water |
| | | penetration | penetration |
| | | observed | observed |
| 500 ml | 48 hours | No water | No water |
| | | penetration | penetration |
| | | observed | observed |

A barrier face can be described in terms of its breathability, which can be expressed in terms of the number of grams of water vapor that can pass through a square meter of the fabric in a 24 hour period. A barrier face can have a breathability (per ASTM E96-BW) of, e.g., from 0 to about 5000 g/m²/day, or from about 5,000 to 10,000 g/m²/day, or even from about 10,000 to 20,000 or even 30,000 g/m²/day. A barrier face may also have RET rating of between about 3 and about 6; it may also have a RET (Hohenstein) rating of less than about 3.

Embodiment 20. The protector article of any one of Embodiments 1-19, wherein the water-resistant biodegradable material comprises a plant-derived polymer or monomer. Example such materials include, without limitation, Mater — BI™ and PLA.

Embodiment 21. The protector article of any one of Embodiments 1-20, wherein the water-resistant biodegradable material is characterized as being plant-derived.

Embodiment 22. The protector article of any one of Embodiments 1-21, wherein the protector article is characterized as a bedsheet that fits over a mattress. An article can be characterized as configured to fit over a United States twin (regular or long), double, full, queen, king, or California king size mattress.

Embodiment 23. The protector article of any one of Embodiments 1-21, wherein the protector article is characterized as a pillowcase.

Embodiment 24. The protector article of any one of Embodiments 1-21, wherein the protector article is characterized as a seat cover.

Embodiment 25. The protector article of any one of Embodiments 1-21, wherein the protector article is characterized as a garment. Exemplary such garments include, e.g., aprons, gowns, mitts, gloves, and the like.

Embodiment 26. The protector article of any one of Embodiments 1-21, wherein the comfort face of the protector article remains essentially visually unchanged following about 7-10 days of standard usage of 8 hours per day.

Embodiment 27. A method, comprising disposing a protector article according to any one of Embodiments 1-21 over a mattress.

Embodiment 28. A method, comprising disposing a protector article according to any one of Embodiments 1-21 over a pillow.

Embodiment 29. A method, comprising disposing a protector article according to any one of Embodiments 1-21 over a seat.

Embodiment 30. A method, comprising fabricating a protector article according to any one of Embodiments 1-26.

Embodiment 31. A method, comprising: forming an article that comprises a plurality of sheets overlaid one to the next, a sheet comprising a comfort face and a barrier face, the comfort face comprising a biodegradable non-woven spunlace material, the barrier face comprising either a water-resistant or water repellent or water proof biodegradable material, at least one of the plurality of sheets optionally being releasably attachable to the article.

Suitable sheets and materials are described elsewhere herein.

Embodiment 32. The method of Embodiment 31, further comprising forming a sheet.

Embodiment 33. The method of Embodiment 32, wherein the forming comprises assembling together the comfort face and the barrier face.

Embodiment 34. The method of Embodiment 33, wherein the assembling comprises laminating. Laminating can be performed at, e.g., from about 60 to about 95 deg. C. The dwell time of the lamination can be from about 0.1 to about 1,000 seconds, or from about 1 to about 500 seconds, or from about 10 to about 200 seconds, and all intermediate values and subranges.

Embodiment 35. A protector article, comprising: a plurality of sheets overlaid one to the next, a sheet comprising a comfort face and a barrier face, the comfort face comprising a nonwoven biodegradable spunlace material derived from cellulose and a polysaccharide, the barrier face comprising a water-resistant biodegradable material, the water-resistant biodegradable material optionally being porous, and at least one of the plurality of sheets being releasably attached to the article; and a sleeve, a strap, a band, a skirt having an adjustable perimeter and optionally comprising a biodegradable elastic material, or any combination thereof.

1. A protector article, comprising:
a plurality of sheets overlaid one to the next, the plurality of sheets comprising a bottommost sheet,
a sheet comprising a comfort face and a barrier face,
the comfort face comprising a biodegradable spunlace material,
the barrier face comprising a water-resistant biodegradable material, and
at least one of the plurality of sheets being releasable from the article.

2. The protector article of claim 1, further comprising a sleeve, a strap, a band, a skirt having an adjustable perimeter, or any combination thereof, the skirt optionally comprising a biodegradable elastic or non-elastic material.

3. The protector article of claim 2, wherein the sleeve, the strap, the band, the skirt having an adjustable perimeter, or any combination thereof is secured to the bottommost sheet.

4. The protector article of claim 1, further comprising a base layer.

5. The protector article of claim 4, further comprising a sleeve, a strap, a band, a skirt having an adjustable perimeter, or any combination thereof.

6. The protector article of claim 5, wherein the sleeve, the strap, the band, the skirt having an adjustable perimeter, or any combination thereof is secured to the base layer.

7. The protector article of claim 1, wherein at least two of the plurality of sheets are secured to one another by gluing, snaps, buttons, hook-and-loop fasteners, heat-sealing, clamping, or any combination thereof.

8. The protector article of claim 1, wherein at least one of the plurality of sheets is attached to the protector article via one or more perforated linkages, one or more hook-and-loop fasteners, or one or more stitched linkages.

9. The protector article of claim 1, wherein at least one of the plurality of sheets is attached to the protector article via one or more adhesive linkages.

10. The protector article of claim 1, wherein at least a portion of the biodegradable spunlace material is characterized as plant-derived.

11. The protector article of claim 10, wherein at least a portion of the biodegradable spunlace material is derived from one or more of corn, seaweed, and agave.

12. The protector article of claim 10, wherein at least a portion of the biodegradable spunlace material comprises polycaprolactone, starch, polylactic acid, or any combination thereof.

13. The protector article of claim 1, wherein the biodegradable spunlace material comprises one or more of cotton, viscose, bamboo, hemp, flax, or any combination thereof.

14. The protector article of claim 1, wherein the comfort face is characterized as having a GSM of from about 18 to about 60 g/m².

15. The protector article of claim 1, wherein the comfort face is characterized as comprising at least a first fiber population and a second fiber population, the first fiber population differing from the second fiber population in at least one of size and composition.

16. The protector article of claim 1, wherein the comfort face is characterized as having a static coefficient of friction of from about 0.01 to about 0.5.

17. The protector article of claim 1, wherein the comfort face is characterized as having a softness.

18. The protector article of claim 1, wherein the barrier face is characterized as porous.

19. The protector article of claim 1, wherein the barrier face is characterized as having a waterproof rating of from about 0 to about 50,000, a breathability of from about 0 to about 30,000 g/m²/day, or any combination thereof.

20. The protector article of claim 1, wherein the water-resistant biodegradable material comprises a plant-derived monomer or polymer.

21. The protector article of claim 1, wherein the water-resistant biodegradable material is characterized as being plant-derived.

22. The protector article of claim 1, wherein the protector article is characterized as a bedsheet that fits over a mattress.

23. The protector article of claim 1, wherein the protector article is characterized as a pillowcase.

24. The protector article of claim 1, wherein the protector article is characterized as a seat cover.

25. The protector article of claim 1, wherein the protector article is characterized as a garment.

26. The protector article of claim 1, wherein the comfort face of the protector article remains essentially visually unchanged following about 7-10 days of standard usage.

27. A method, comprising disposing a protector article according to claim 1 over a mattress.

28. A method, comprising disposing a protector article according to claim 1 over a pillow.

29. A method, comprising disposing a protector article according to claim 1 over a seat.

30. A method, comprising fabricating a protector article according to claim 1.

31. A method, comprising:
forming an article that comprises a plurality of sheets overlaid one to the next,
a sheet comprising a comfort face and a barrier face,
the comfort face comprising a biodegradable non-woven spunlace material,
the barrier face comprising a water-resistant biodegradable material,
at least one of the plurality of sheets optionally being releasable from the article.

32. The method of claim 30, further comprising forming a sheet.

33. The method of claim 32, wherein the forming comprises assembling together the comfort face and the barrier face.

34. The method of claim 33, wherein the assembling comprises laminating.

35. A protector article, comprising:
a plurality of sheets overlaid one to the next,
a sheet comprising a comfort face and a barrier face,
the comfort face comprising a biodegradable non-woven spunlace material derived from cellulose and a polysaccharide,
the barrier face comprising a water-resistant biodegradable material, the water-resistant biodegradable material optionally being porous, and
at least one of the plurality of sheets being releasable from the article; and
a sleeve, a strap, a band, a skirt having an adjustable perimeter, or any combination thereof, the skirt optionally comprising a biodegradable elastic material.
